(19) United States
(12) Patent Application Publication
(10) Pub. No.: US 2005/0015123 A1
(43) Pub. Date: Jan. 20, 2005

(54) Endovascular treatment of a blood vessel using a light source

(76) Inventor: Dilip Y. Paithankar, Natick, MA (US)

Correspondence Address: PROSKAUER ROSE LLP, ONE INTERNATIONAL PLACE 14TH FL, BOSTON, MA, 02110, US

(21) Appl. No.: 10/882,089

(22) Filed: Jun. 30, 2004

Related U.S. Application Data
(60) Provisional application No. 60/483,737, filed on Jun. 30, 2003.

(51) Int. Cl. A61N 5/06; A61F 7/12

(52) U.S. Cl. 607/88; 607/113

(57) ABSTRACT

A method and apparatus to endovascularly treat blood vessels using a light beam that causes minimal collateral damage to surrounding tissue is described. The technique can improve the appearance of a blood vessel and/or reduce its size, as well as relieve other medical symptoms. The wavelength of the light beam can be selected so as to heat one or more chromophores either inside the blood vessel or within the blood vessel wall itself. Access to the vein lumen of the targeted blood vessel can be obtained via an optical fiber inserted into the blood vessel through a catheter.

### CROSS-REFERENCE TO RELATED APPLICATION

This application claims the benefits of and priority to U.S. Provisional Patent Application Ser. No. 60/483,737 filed on Jun. 30, 2003, which is owned by the assignee of the instant application and the disclosure of which is incorporated herein by reference in its entirety.

### GOVERNMENT RIGHTS

This invention was made with government support under Contract No. 1 R43 HL076931 01 awarded by the National Institute of Health. The government may have certain rights in the invention.

### FIELD OF THE INVENTION

This invention relates generally to the field of vascular medicine and dermatology, and more particularly to methods and apparatus for endovascularly treating blood vessels using a light source.

### BACKGROUND OF THE INVENTION

Dilated blood vessels present significant medical and cosmetic problems affecting a large fraction of the population, especially women. For example, varicose veins are a common condition, particularly in older women. A cause of varicose veins is reflux and pooling of blood due to incompetent venous valves, which can lead to dilated, tortuous, bulging veins. Varicose veins can cover a large area of skin and be quite unattractive. In addition, they can become quite uncomfortable, causing swelling, fatigue, and pain in the legs, as well as clogging blood vessels and causing ulcers. Physiological problems, such as pain and complications, are not associated with all dilated blood vessels, though, and, medical treatment is not always required. For example, spider veins, which are dilations of small, superficial blood vessels, and small varicose veins can be treated for a purely cosmetic benefit.

Conventional treatments for vein disorders include removal of the vein by stripping or ambulatory phlebectomy, or closing of the vein by sclerotherapy. Treatment of a varicose vein with either laser energy or RF energy is another option. Typically, laser treatment involves laser irradiation within the lumen of the vein, e.g., the greater saphenous vein, by inserting an optical fiber through the surface of the skin and targeting blood as the chromophore, which may result in undesirable collateral damage to the surrounding tissue and skin.

For example, current laser based treatments employing the wavelengths of 810 nm, 940 nm, and 980 nm are associated with post-operative bruising and tenderness. These laser wavelengths are absorbed principally by hemoglobin in the blood. The heat is transferred to the vein wall causing thermal damage, but the laser irradiated blood can reach very high temperatures. It has been suggested that the underlying treatment mechanism is laser induced indirect local heat injury of the inner vein wall by steam bubbles originating from boiling blood. [See, Proebstle et al., J. Vasc. Surg. 35, 729, (2002).] Currently used laser based techniques targeting blood can lead to uncontrolled heating and thermal injury to the vein wall and to tissue outside the wall, possibly by boiling blood, resulting in the post-operative bruising and tenderness.

Accordingly, a need has arisen in the art for an improved method and apparatus for endovascularly treating blood vessels using a light source that substantially eliminates the deficiencies of prior techniques described above. In particular, there is a need for a method and apparatus that provides selective, controlled heating of the vein wall and minimal heating outside the vein wall, thus minimizing undesirable collateral damage. This can be performed by a proper choice of the wavelength of the light.

### SUMMARY OF THE INVENTION

The invention features a method and apparatus to endovascularly treat blood vessels using a light beam that causes minimal collateral damage to surrounding tissue. In some embodiments, the treatment is therapeutic. It can be used to relieve physiological problems or symptoms associated with dilated blood vessels. In other embodiments, the treatment is purely cosmetic. For example, the treatment technique can be used to improve the characteristics of the skin for purely cosmetic purposes, e.g., improving the appearance of the blood vessel and/or reducing its size, when there is no medical necessity to undergo the treatment.

The wavelength of the light beam can be selected to heat one or more chromophores (e.g., water) either within the blood vessel wall or inside the blood vessel. Access to the vein lumen of the targeted blood vessel can be obtained by a catheter, which can include a fiber optic insert. The treatment can allow one to directly target a wall of the blood vessel, which decreases thermal damage to surrounding tissue as compared to laser irradiation above the skin which may cause undesirable damage to skin and tissue that the light penetrates or as compared to heating the blood within the vessel.

In addition, by contacting the wall of the blood vessel directly or by removing blood from the blood vessel, the treatment avoids coagulation of the blood during treatment. Fibrosis of blood vessel wall has been shown to be preferable to blood coagulation as a way of treating blood vessel. Treating a blood vessel endovascularly can reduce the energy input needed to cause the desired response and therapeutic effect, since the surrounding skin tissue does not need to be penetrated to reach the desired target blood vessel. Furthermore, by targeting water, longer wavelengths of light (greater than about 1,160 nm) can be used, and the penetration depth of the light beam can be adjusted. By controlling the penetration depth, collateral damage to surrounding tissue can be controlled and minimized.

In one aspect, the invention is directed to a method of treating a blood vessel. In some embodiments, the treatment is therapeutic and can be used to relieve physiological problems or symptoms associated with dilated blood vessels. The method can also be used to improve the visual appearance of a blood vessel visible through the skin. In one embodiment, the method includes providing a beam of light having a wavelength longer than about 1,160 nm and delivering endovascularly the beam of light to a wall of a targeted blood vessel.

In one embodiment, the method includes delivering the beam of light to a target chromophore (e.g., water) in the wall of the targeted blood vessel. In one embodiment, the method includes reducing the size of the targeted blood vessel. In various embodiments, the method includes heating the target chromophore to a temperature below about 80° C. In some embodiments, the method can include substantially removing blood from at least a portion of the targeted blood vessel before delivering endovascularly the beam of light to the wall of the targeted blood vessel.

In various embodiments, the beam of light can be delivered using an optical fiber. The optical fiber can include a diffusing tip connectable to the optical fiber. In one embodiment, the optical fiber can be in communication with a pullback device for positioning the optical fiber. The pullback device can withdraw the optical fiber from the targeted blood vessel at a rate of between about 0.5 mm/s and about 2 mm/s.

In various embodiments, the beam of light has a wavelength between about 1160 nm and about 2600 nm. In some embodiments, the wavelength of the beam of light is between about 1300 nm and about 1560 nm. In one detailed embodiment, the beam of light has a wavelength of about 1450 nm. In another detailed embodiment, the beam of light has a wavelength of about 2100 nm.

The fluence of the beam of light can be between about 3 J/cm² and about 100 J/cm². In various embodiments, the power of the beam of light is between about 0.5 W and about 5 W. The irradiation time of the beam of light can be between about 0.2 s and about 10 s. In various embodiments, the penetration depth of the beam of light is between 0.05 mm and about 2.0 mm. In one detailed embodiment, the penetration depth of the beam of light is about 300 μm.

In another aspect, the invention features a method of treating a blood vessel. The method includes providing a beam of light having a wavelength longer than about 1,160 nm. A light-absorbing medium is introduced adjacent a wall of a targeted blood vessel, and the beam of light is delivered endovascularly to the light-absorbing medium, which absorbs at least one wavelength of the beam of light.

In yet another aspect, the invention features an apparatus for treating a blood vessel. The apparatus includes a light source providing a beam of light having a wavelength longer than about 1,160 nm, and a delivery system for introducing a light-absorbing medium adjacent a wall of a targeted blood vessel. The apparatus also includes an optical fiber for delivering endovascularly the beam of light to the light-absorbing medium in the targeted blood vessel.

In still another aspect, the invention is directed to an apparatus for treating a blood vessel. The apparatus includes a means for providing a beam of light having a wavelength longer than about 1,160 nm, a means for introducing a light-absorbing medium adjacent a wall of a targeted blood vessel, and a means for delivering endovascularly the beam of light to the light-absorbing medium.

In another aspect, the invention provides a kit for treating, either therapeutically or cosmetically treating, a blood vessel. The kit includes a light source providing a beam of light having wavelength longer than about 1,160 nm and an optical fiber for delivering endovascularly the beam of light to a wall of a targeted blood vessel. The kit also includes instructions for using the light source and the optical fiber to improve the appearance of the targeted blood vessel by reducing its size.

Other aspects and advantages of the invention will become apparent from the following drawings, detailed description, and claims, all of which illustrate the principles of the invention, by way of example only.

### BRIEF DESCRIPTION OF THE DRAWINGS

The advantages of the invention described above, together with further advantages, may be better understood by referring to the following description taken in conjunction with the accompanying drawings. In the drawings, like reference characters generally refer to the same parts throughout the different views. The drawings are not necessarily to scale, emphasis instead generally being placed upon illustrating the principles of the invention.

FIG. 1 is a block diagram showing an illustrative embodiment of an apparatus for treating a blood vessel according to the invention.

FIGS. 2A-2C are cross-sectional views of exemplary embodiments of optical fiber tips according to the invention.

FIG. 3 is a cross-sectional view of a leg being treated using a technique of the invention.

FIG. 4 is a graph of optical penetration depth versus wavelength.

FIG. 5 is a cross-sectional view of a leg being treated using a technique of the invention including a light-absorbing medium.

FIGS. 6A, 7A, and 8A are graphs showing the temperature plotted versus depth through the center of the treated spot at the end of the laser pulse for a fluence of 12 J/cm² and irradiation times of 1 s, 2 s, and 5 s, respectively.

FIGS. 6B, 7B, and 8B are graphs showing the thermal damage plotted versus depth through the center of the treated spot at the end of the laser pulse for the same fluence and irradiation times as the corresponding FIGS. 6A, 7A, and 8A.

FIGS. 9-11 are photographs of histology slides of cross-sections of pig aortas that were treated with laser radiation using a technique of the invention.

FIG. 12 is a photograph of a histology slide of a cross-section of a human vein prior to being treated with laser radiation.

FIG. 13 is a photograph of a histology slide of a cross-section of a human vein treated with laser radiation using a technique of the invention.

### DESCRIPTION OF THE INVENTION

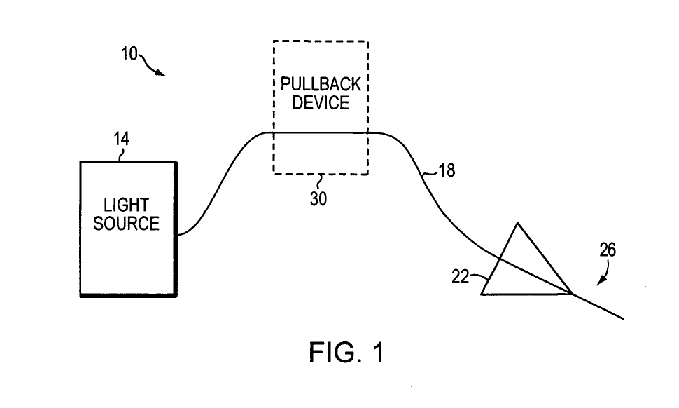

FIG. 1 depicts an illustrative embodiment of an apparatus 10 for delivering a beam of light endovascularly to a target blood vessel. The apparatus 10 can include a light source 14, an optical fiber 18, and a catheter 22. The light source 14 is in optical communication with the optical fiber 18 for delivering the beam of light. The catheter 22 is adapted to be insertable into a blood vessel of a patient. The optical fiber 18 can be inserted into the catheter for access to the blood vessel of the patient.

In one embodiment, a guidewire or pullwire is coupled to an end portion 26 of the optical fiber 18 for positioning the optical fiber 18 inside the blood vessel. In this embodiment, the guidewire or pullwire can be inserted through the catheter 22 as well. In an alternative embodiment, the guidewire or pullwire can be connectable to the catheter 22. In yet another embodiment, the guidewire or pullwire can be connectable to both the optical fiber 18 and the catheter 22 for positioning of the devices within a target blood vessel.

In various embodiments, the apparatus 10 includes a pullback device 30 for positioning the optical fiber 18. The guidewire or pullwire may be elements of the pullback device 30. The pullback device 30 can improve the control of delivery of the energy from the light source 14, which can help eliminate unwanted side effects. In various embodiments, the pullback device 30 can be automated and/or motorized. In some embodiments., the pullback device 30 operates at a constant speed, and in other embodiments, the pullback device 30 can be stepped at an irregular increment. Operation of the pullback device 30 will be described in more detail below.

In various embodiments, the optical fiber 18 can have a diameter between about 50 μm and about 1000 μm. In one embodiment, the optical fiber 18 has a diameter of between about 200 μm to about 800 μm. In another embodiment, the optical fiber 18 has a diameter of between about 300 μm to about 600 μm.

The optical fiber 18 can be a single fiber, or a bundle of fibers. In some embodiments, the optical fiber 18 is coated to protect its integrity from the environment of the body or the blood. In various embodiments, the optical fiber 18 can have a rounded tip to prevent piercing of the wall of the target vessel. The optical fiber 18 can have a tip that emits diffuse light.

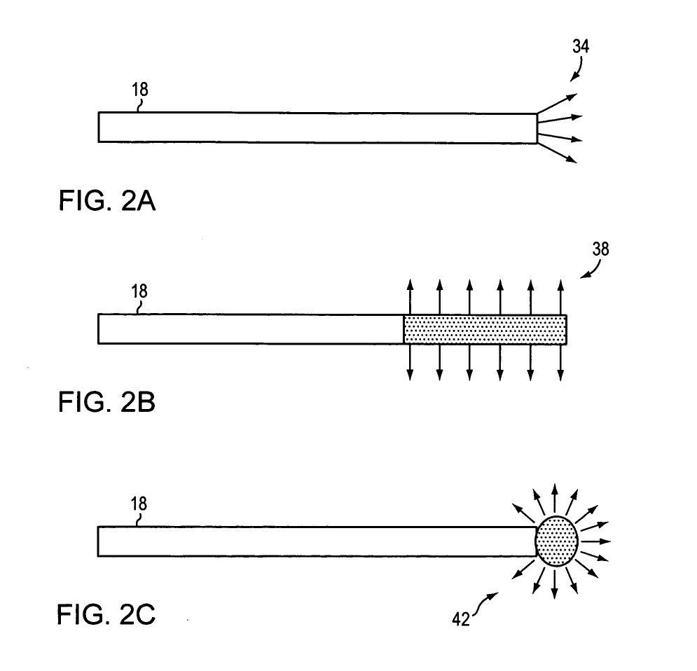

FIGS. 2A-2C depict exemplary embodiments of tips for an optical fiber. During treatment, a target blood vessel is compressed on the optical fiber with tumescent anesthesia to deliver light to the vessel wall. Alternatively, a guidewire can be used to manipulate the end portion 26 of the optical fiber 18 so that light can be directed substantially off-axis from the longitudinal axis of the optical fiber 18. For example, using a guidewire, the end portion 26 of the optical fiber 18 can be bent to form an angle of between about 0° and about 90° with relation to the longitudinal axis of the optical fiber 18. In some embodiments, other means known in the art are used to manipulate the end portion 26 of the optical fiber 18.

In various embodiments, non-diffusing fiber tips direct energy substantially along the longitudinal axis of the optical fiber 18 to deliver the light to the vessel wall. In other embodiments, diffusing fiber tips can be used to deliver light to the vessel wall. Using diffusing fiber tips, light can be directed laterally from the end portion 26 of the optical fiber 18, which can allow more precise heating and destruction of the vein wall and provide a more uniform and predictable shrinkage of the vein. Furthermore, a guidewire can also be used to manipulate the end portion 26 of the optical fiber 18.

FIG. 2A shows an illustrative embodiment of an optical fiber 18 with a bare fiber tip 34. The bare fiber tip 34 can be the simplest and least expensive design, and can be obtained by cleaving an optical fiber. In an embodiment using a tumescent anesthesia around the vein during treatment, the vein is highly compressed and collapsed around the bare fiber tip 34. The light from the bare fiber tip 34 can coagulate the tissue being irradiated. The arrows approximate the propagation of the light from the fiber tip.

FIG. 2B shows an illustrative embodiment of an optical fiber 18 with a linear diffuser tip 38. The light from this tip 38 is delivered laterally to the vein wall causing heating and coagulation. FIG. 2C depicts an illustrative embodiment of a spherical ball-type diffuser tip 42, which emits light radially from the fiber tip. The diffuser tip 38 or 42 can include a scattering material, such as a polymer cover or a ceramic cover, to, for example, overcome the index of refraction matching properties of the optical fiber and the adjacent fluid or tissue. The diffuser tips 38 and 42 are more expensive than bare fiber tip 34, but may provide better control of the light delivered.

In one detailed embodiment, after prolonged use (e.g., operation at a CW optical power of 5 W for 8 hours), an optical fiber tip experiences less than a 95% drop in transmission, has no discernible changes to its appearance upon visual inspection, and exhibits no increase in temperature during irradiation. Preferably, the optical fiber tip delivers full thickness damage to the vein wall in the absence of explosive ablation or vein perforations. In one embodiment, the fiber tip can include a disposable sheath placed over the tip. In another embodiment, the fiber tip includes an air gap.

In various embodiments, the diffuser tip 38 or 42 can be permanently or removably affixed to the optical fiber 18. The diffuser tip 38 or 42 can be affixed using an adhesive, a bonding agent, a joining compound, an epoxy, a clip, a thread, other suitable mechanical connection or attachment means, or some combination thereof.

In various embodiments, the light can be coherent or incoherent light, or a combination. The light can be monochromic or otherwise. In some embodiments, the source is coherent and can be, for example, a pulsed, scanned, or gated continuous wave (CW) laser. Exemplary light sources include, but are not limited to diode lasers, doped fiber lasers, solid state lasers, and flashlamps. Other sources of light/energy include acoustic waves, microwaves, and RF radiation. For the purposes of this disclosure, these radiation/energy sources are considered to be light.

A suitable diode laser is an InGaAsP laser (about 1450 nm). Suitable solid state lasers include, but are not limited to, Nd:YAG (about 1320 nm), Nd:YAP (about 1340 nm), Er:YAP (about 1660 nm), Cr, Tm, Ho:YSGG (about 2100 nm), Er:YAG (about 1640 nm and/or about 1770 nm), Cr, Tm, Ho:YAG (about 2010 nm to about 2130 nm), Nd:YVO4 (about 1342 nm), Er:Glass (about 1535 nm), and Er:YLF (about 1230 nm and/or about 1730 nm).

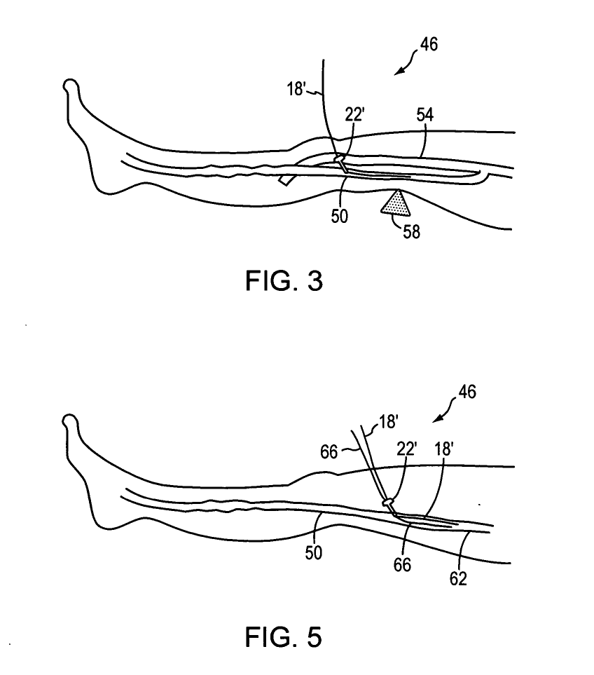

FIG. 3 depicts a leg 46 with a target blood vessel 50 and a healthy blood vessel 54. To treat the target blood vessel 50, blood can be removed from the vessel by compression of the vessel at or near the target blood vessel 50. This can be performed by such methods as elevation of the leg 46, compression bandaging, and/or direct application of pressure by a compression device 58, such as a hand, fingers, or an object. Blood can also be removed by using a tumescent anesthesia, e.g., a percutaneous injection of a solution containing lidocaine under pressure. A catheter 22′ can be inserted into the leg 46, and an optical fiber 18′ can be guided into the target blood vessel 50 via the catheter 22′. Compression, as described above, also ensures sufficient contact between the target blood vessel 50 and the optical fiber 18′.

Blood vessels of various diameters, e.g., about 1 mm to about 6 mm, can be treated using the techniques of the invention, although larger and smaller diameter vessels can be treated as well. It will be appreciated that some of the uses of the apparatus and technique described herein relate to therapeutic treatments. However, there are also uses that relate solely to cosmetic treatments. Not all dilated blood vessels cause physiological problems. For example, some smaller, surface-lying veins do not cause any difficulty or discomfort for a person—they just appear ugly or unsightly. When these veins are treated, it is for purely cosmetic reasons, i.e., there is no therapeutic benefit.

In accordance with the invention, a patient can have a lower leg treated for varicose veins, with some veins treated where the veins are in need of therapeutic treatment (because they do ache/cause physiological difficulties), and other veins treated purely for cosmetic reasons (where the veins do not cause any discomfort or physiological problem). In one exemplary embodiment, the target blood vessel 50 can be described as an unsightly blood vessel, and the healthy blood vessel 54 can be a blood vessel with a normal visual appearance.

As described above, the optical fiber 18 or 18′ is connected to a light source (14 in FIG. 1; not shown in FIG. 3), which provides a beam of light with a wavelength or energy that targets a chromophore in the blood vessel or in the blood vessel wall. In one detailed embodiment, the chromophore is water. In various other embodiments, the chromophore is collagen, a blood cell, hemoglobin, plasma, or other component of blood. In an embodiment where a component of blood absorbs the radiation and heats up, the chromophore transfers the energy to the blood vessel wall and causes irreversible thermal injury to the blood vessel.

The wavelength of light can be selected to match the depth of light absorption (also called penetration depth), and therefore heat to the vein wall thickness. In various embodiments, the wavelength of the light can be selected so that the light is principally absorbed within a distance of about 2.0 mm or less from the surface of the wall of the target vessel. A commonly accepted definition of penetration depth of light is the depth at which the fluence reaches 36.8% of the value at the surface upon exponential decay with depth. Preferably, the light wavelength is selected so that the penetration depth is between about 0.05 mm and about 2.0 mm. More preferably, the wavelength is selected so that the penetration depth is greater than about 0.1 mm and less than about 2.0 mm. The invention also contemplates a penetration depth less than about 0.05 mm and greater than about 2.0 mm.

Light causes heating both above and below this depth. To examine the appropriate wavelengths, a simplifying assumption is made that the optical penetration depth (OPD) of light is given by Equation (1) after application of the diffusion approximation:
OPD=[3μₐ{μₛ(1−g)+μₐ}]^{-1/2} (1)
For Equation (1), see, chapter by L. O. Svaasand, “Physics of Laser-Induced Hyperthermia,” at page 778 in book “Optical-Thermal Response of Laser-Irradiated Tissue,” edited by A. J. Welch and M. J. C. van Gemert, Plenum Press: New York and London (1995), the entire disclosure of which is incorporated herein by reference.

In the Equation (1), μₐ and μₛ are the absorption coefficient and scattering coefficient, respectively, and g is the anisotropy factor of the irradiated tissue. The absorption coefficient of tissue can be taken to be 0.7 times the water absorption coefficient since tissue water content is approximately 70%. This is a reasonable assumption because in the wavelength range of interest, water is the primary absorbing component. The values of μₛ and g can be taken to be 120 cm⁻¹ and 0.9, respectively, as a simplifying assumption. In actuality, these do depend on the wavelength. With the above values, OPD is calculated for various wavelengths from the water absorption coefficients versus wavelength data.

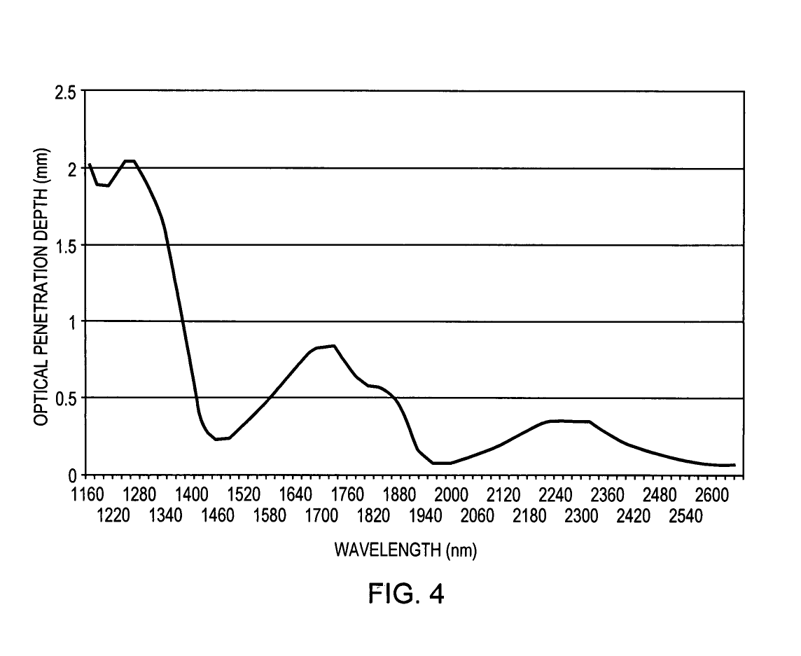

FIG. 4 shows a graph of optical penetration depth versus wavelength. The absorption coefficient data was taken from Hale and Querry “Optical constants of water in the 200 nm to 200 μm wavelength region,” Applied Optics 12, 555 (1973), the disclosure of which is incorporated herein by reference in its entirety. The advantage of choosing wavelengths where the penetration depth is between about 0.05 mm and about 2.0 mm is that collateral damage to tissue surrounding the target blood vessel, and to the overlying skin, can be minimized, thereby reducing the incidence and severity of side effects, cosmetic or otherwise.

As is shown in FIG. 4, the light may have a wavelength between about 1160 nm and about 2600 nm. In certain embodiments, the light can have a wavelength between about 1320 nm and about 1900 nm. In certain other embodiments, the light has a wavelength between about 1300 nm and about 1560 nm, between about 1840 nm and about 1900 nm, between about 1980 nm and about 2600 nm, between about 1980 nm and about 2140 nm, or between about 2340 nm and about 2600 nm.

The fluence is determined depending on the application and the wavelength used. The fluence can be within the range of about 1.66 J/cm² to about 270 J/cm². In certain embodiments, the fluence is between about 3 J/cm² and about 100 J/cm².

The power of the laser can be between about 0.2 W and about 20 W. In some embodiments, the power is between about 5 W and about 10 W.

The irradiation time can be between about 0.01 s and about 30 s, although the irradiation time can be longer or shorter depending on the application. In some embodiments, the irradiation time can be between about 0.2 s to about 10 s.

During treatment, the catheter can be used to facilitate placement of the optical fiber. The catheter is inserted into the target blood vessel, and the optical fiber is fed through the catheter and positioned adjacent to the portion of the target blood vessel to be treated. A compression device, if used, is positioned to apply sufficient compression to the target vessel to remove the blood. An imaging device, such as an ultrasound or an infrared camera, may be used to monitor the placement of the optic fiber, particularly its tip. In an alternative embodiment, a catheter is not used. An incision is made near the target, and the optical fiber is inserted directly into the target blood vessel.

During treatment, the optical fiber is inserted, either with or without the catheter, and the light is delivered to the portion of the vessel to be treated. Without removing the optical fiber from the leg, the fiber and the compression device may be moved within the target blood vessel to treat another portion. This process is repeated until the target blood vessel is sufficiently therapeutically injured, preferably to cause the vessel to undergo fibrosis and disappear. Alternatively or in addition to, the optical fiber, with or without the catheter, is inserted into a plurality of locations along the target blood vessel. This technique may be used if a longer vessel is to be treated. The optical fiber may still be repositioned within the target blood vessel to treat more than one portion of the vessel for each position of the catheter or incision of the leg.

As described above, to position the optical fiber and the catheter, a pullback device can be used. For example, the pullback device can be started so that the optical fiber and/or catheter is withdrawn from the target blood vessel for about 2 mm prior to turning the light source on. In various embodiments, the pullback device can be motorized and or automated so that the optical fiber and/or catheter can be withdrawn from the target blood vessel at a constant rate.

Suitable withdrawal rates can be between about 0.05 mm/s and about 10 mm/s, although higher and lower rates can be used depending on the application. In some embodiments, the withdrawal rate is between about 0.5 mm/s and about 2 mm/s. This translates into a total time of treatment for a 40 cm vein of between about 13.3 min. and 3.3 min.

Upon heating a vein with a light source, the water in the tissue can reach the boiling point of 100° C. Further heating can cause steam bubble formation in an explosive ablation process that can lead to removal of tissue and possibly perforation of the vein wall. For the technique of the invention, it is preferable but not necessary that the fluence used is lower than this ablation threshold. Ablation and vein perforation can be monitored by using a detector to measure the temperature of the surface irradiated. The detector can be a thermocouple device or an imaging device. In one embodiment, the imaging device can have a fast response time (e.g., less than about 1 ms). In one detailed embodiment, the detector is a commercially available liquid nitrogen cooled IR camera.

One exemplary light source is an InGaAsP diode laser providing a wavelength of 1450 nm. The peak power of the laser is between about 0.5 W and about 10 W. The laser can be operated in either CW or pulsed mode. Pulse durations for this laser can be between about 10 ms and about 10 s. In one detailed embodiment, the light source is a laser diode system that includes a linear array of diode lasers operating at 1450 nm. The maximum CW optical power can be about 5 W. Light from the InGaAsP diode laser can be coupled into an optical fiber with a 400 μm core. The distal end of the fiber can be a cleaved bare end or a diffuser tip.

At this chosen wavelength, the laser energy is principally absorbed by water in the vein wall causing photothermal irreversible injury to the vein wall alone, while sparing tissue outside the vein wall and without causing perforations. For a 1450 nm wavelength laser, detailed Monte Carlo calculations show that the (1/e) depth, i.e., depth at which the fluence reaches 36.8% of the fluence at the surface, is 439 μm. Also, heat transfer and damage calculations, examples of which follow below, show the damage to be on the order of 300 μm. Thus, this wavelength produces thermal damage matched to the vein wall thickness. (Typical vein wall thicknesses can be between about 100 μm and about 350 μm.)

Furthermore, light of a wavelength that is strongly absorbed by tissue is absorbed within a small depth and results in a large increase in temperature and possibly ablation. In contrast, a wavelength weakly absorbed by tissue is absorbed to a much larger depth, thereby causing deeper heating. As described above, the 1450 nm light has the property of approximately matching the depth of heating to the vein wall thickness. This can lead to a highly effective treatment with minimal side effects and no high peak temperatures leading to explosive ablation, so perforation of the vein and resulting bruising and tenderness may be absent. The control of the heating process and the zone of thermal damage provides advantages over prior treatment techniques, and a 1450-nm diode laser may be more attractive than a flashlamp pumped Nd:YAG laser due to the simplicity, lower cost, and higher efficiency of the diode laser.

According to another illustrative embodiment, a light-absorbing medium is introduced adjacent a wall of a targeted blood vessel and the light-absorbing medium absorbs at least one wavelength of the beam of light. FIG. 5 depicts the leg 46 with the target blood vessel 50, which is treated by inserting the catheter 22′ into the leg 46. The optical fiber 18′ is guided into the target blood vessel 50 via the catheter 22′. The target vessel 50 is irrigated with a light-absorbing medium 62, which is introduced by a light-absorbing medium delivery system 66.

The delivery system 66 for the light-absorbing medium 62 may include a pump, biocompatible tubing, and ports for delivery of the fluid. The light-absorbing medium delivery system, or a portion thereof, can be insertable into the catheter 22′ for delivery of the light-absorbing medium 62. Furthermore, the catheter may be connected to a device that controls delivery of the light-absorbing medium and light through the optical fiber.

Suitable light-absorbing mediums include, but are not limited to, aqueous solutions such as saline solution or an aqueous solution having a sclerotising agent. Light delivered by the optical fiber heats the water in the medium, and that heat is transferred to the wall of the target blood vessel causing irreversible thermal injury to the vessel. Preferably, the light-absorbing medium is heated to between about 60° C. and about 95° C.

The use of a light-absorbing medium permits uniform application of the thermal energy, which reduces the probability of application of excess heat to individual spots of the target blood vessel or to surrounding tissue. In addition, energy delivery through a light-absorbing medium may increase the area of contact, thus reduce treatment time. Alternative sources for delivering energy to heat the light-absorbing medium or a portion of the target blood vessel include radio frequency and ultrasound sources.

The techniques and systems described above are appropriate for blood vessels located within any part of the body endovascularly accessible by a light beam transmitted through a device such as a fiber optic, including the thigh, calf, shin, or near the knee cap. The techniques and systems are also appropriate for blood vessels located in other regions of the body, such as the torso, neck, face, arms, feet, or hands. In addition, the techniques and systems may be used to treat other medical conditions or perform other procedures, such as angioplasty. Blood vessels in the brain, liver, and kidney may be targeted as well.

The invention, in various embodiments, features a kit suitable for use in the endovascular treatment of a blood vessel using a light source. The kit can include a laser and instructions (also known as treatment guidelines) for treating a target blood vessel, e.g., a dilated blood vessel. The instructions can be provided in paper form, for example, in a leaflet, book, or the like, or in electronic form, for example, as a file recorded in a computer readable medium, for example, a diskette, CD-ROM, hard drive or the like. The instructions can include a description of the parameters for performing the treatment. The parameters can be, for example, the laser parameters, such as fluence, irradiance, wavelength, power, depth of penetration, and spot size of the beam of radiation. The parameters can also include, if appropriate, guidelines for the selection of a catheter, an optical fiber, or a suitable withdrawal rate of the catheter or optical fiber. _os Exemplary Calculation of Light and Heat Transport and Depth of Damage

Simulations of light transport and finite difference numerical calculations of temperature distribution can be performed to understand the effect of various fluences with different combinations of irradiation times and power levels. This can help in optimizing the treatment parameters. Monte Carlo simulations can be performed to calculate the light distribution within tissue. Given the light distribution and the absorption coefficient, the heat generated by light due to tissue absorption can be calculated. In addition, numerical heat transfer calculations can be performed to calculate the spatial thermal profiles in tissue. The temperature profiles are indicative of the tissue damage produced.

Detailed calculations of thermal damage can also be done using a kinetic thermal damage model. The kinetic thermal damage model relates the temperature-time history of tissue to the thermal damage, Ω, which is given by Equation (2):

$$Ω = {\int }_{0}^{τ} A exp ( - {E}_{a} / RT ( t ) ) ⅆ t , \qquad ( 2 )$$

where A is a pre-exponential factor, Eₐ is the activation energy, R is the Boltzmann constant, and T(t) is the thermal history as a function of time. A wide range of values for A and Eₐ have been reported for tissue, and the values used for this calculation are 3.1×10⁹⁸ s⁻¹ and 6.28×10⁵ J/mole, respectively. It is assumed that tissue with a damage integral higher than 1 is irreversibly damaged.

Monte Carlo and heat transfer calculations in which appropriate scattering and absorption properties at 1450 nm, as given in Table 1, can be used as input into the model. Heat transfer calculations can be done numerically by a finite-difference method taking into account heating due to light absorption by tissue. In the heat transfer calculations, thermal diffusivity of 0.0008 cm²/s can be used. For this calculation, it can be assumed that there is no heat transfer to the inside surface of the vein wall and that a 6-mm spot on a planar tissue surface is irradiated. The planar geometry is different than the cylindrical geometry of a vein, but this assumption simplifies the calculation. In addition, the tissue absorption coefficient can be assumed to be 70% of the absorption coefficient of water.

TABLE 1
Optical properties used in the Monte Carlo
model for light distribution.

| | Property | | | |
|---|---|---|---|---|
| | Refractive | Absorption | Scattering | Anisotropy |
| Component | Index, n | Coefficient, μₐ | Coefficient, μₛ | factor, g |
| Air | 1 | 0 | 0 | 0 |
| Vein Wall | 1.37 | 20 cm⁻¹ | 120 cm⁻¹ | 0.9 |

An approximate calculation can be presented to make the initial estimate of an appropriate fluence. The critical temperature for thermal damage of aortal tissue has been reported to be about 79° C. The fluence is given by ΔT*ρCₚ/μₐ, if one assumes that there is no heat loss from the heated volume. If the vein wall temperature before irradiation is 35° C., then to achieve a temperature of 100° C., from heat balance, ΔT is 65° C. Using ρcₚ=3.7 J/cm³C and μₐ=20 cm⁻¹, the fluence is calculated to be 12 J/cm².

If the laser energy is delivered over a time longer than the thermal relaxation time, the peak temperature is lower than 100° C. due to heat diffusion. Thus, the fluence of 12 J/cm² can be used as a starting point for detailed calculations. Table 2 below provides the fluence and irradiation time used for eight sets of calculations.

TABLE 2
Fluence, irradiation times, peak temperature, and
damage depth for Monte Carlo and heat
transfer calculations.

| | Calculation Number | | | | | | | |
|---|---|---|---|---|---|---|---|---|
| | 1 | 2 | 3 | 4 | 5 | 6 | 7 | 8 |
| Fluence (J/cm²) | 12 | 12 | 12 | 12 | 12 | 12 | 15 | 20 |
| Irradiation | 0.2 | 0.5 | 1.0 | 2.0 | 5.0 | 10.0 | 10.0 | 10.0 |
| Time (s) | | | | | | | | |
| Peak | 102.2 | 96.1 | 90.0 | 82.8 | 72.7 | 65.0 | 71.9 | 82.7 |
| Temperature | | | | | | | | |
| (° C.) | | | | | | | | |
| Damage | 291 | 303 | 294 | 314 | 313 | 250 | 313 | 548 |
| Depth (μm) | | | | | | | | |

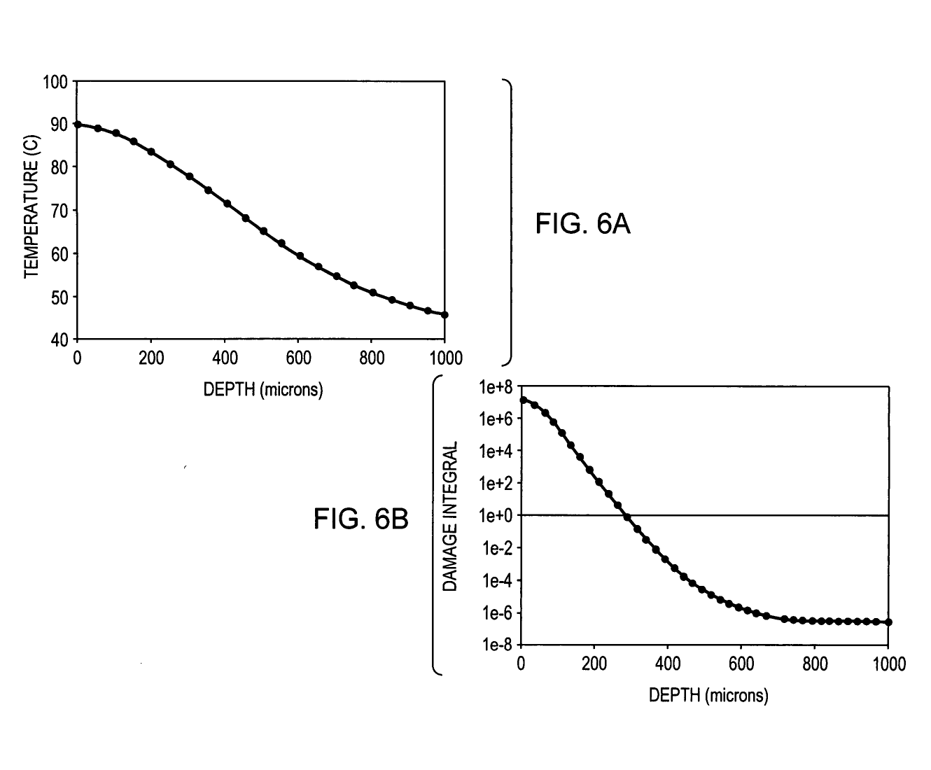

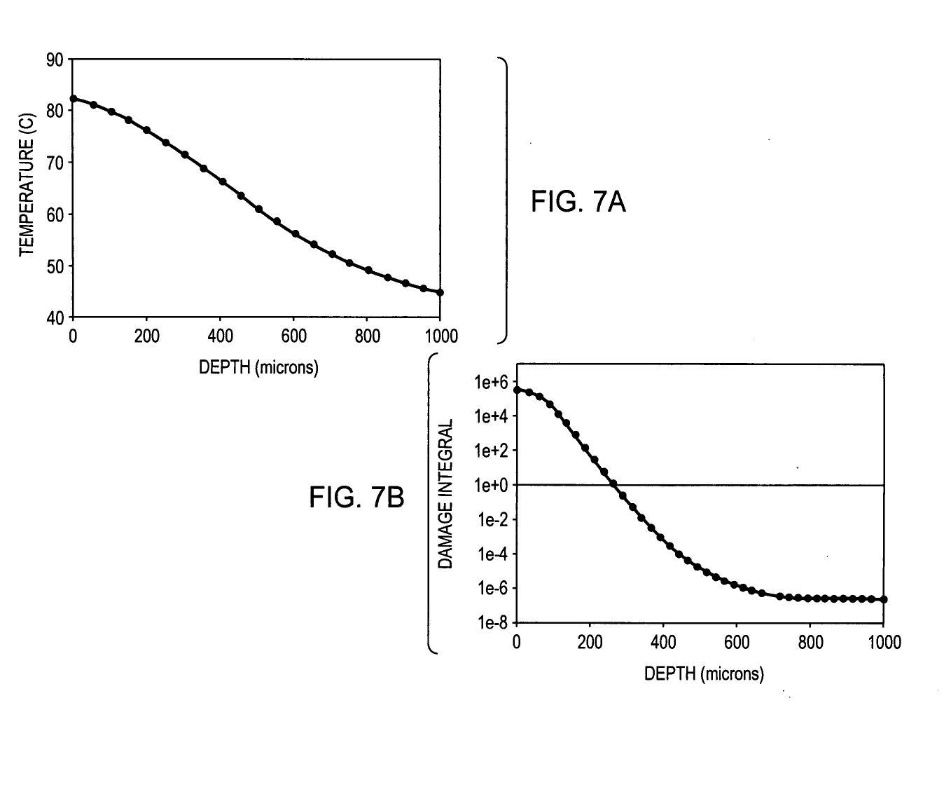

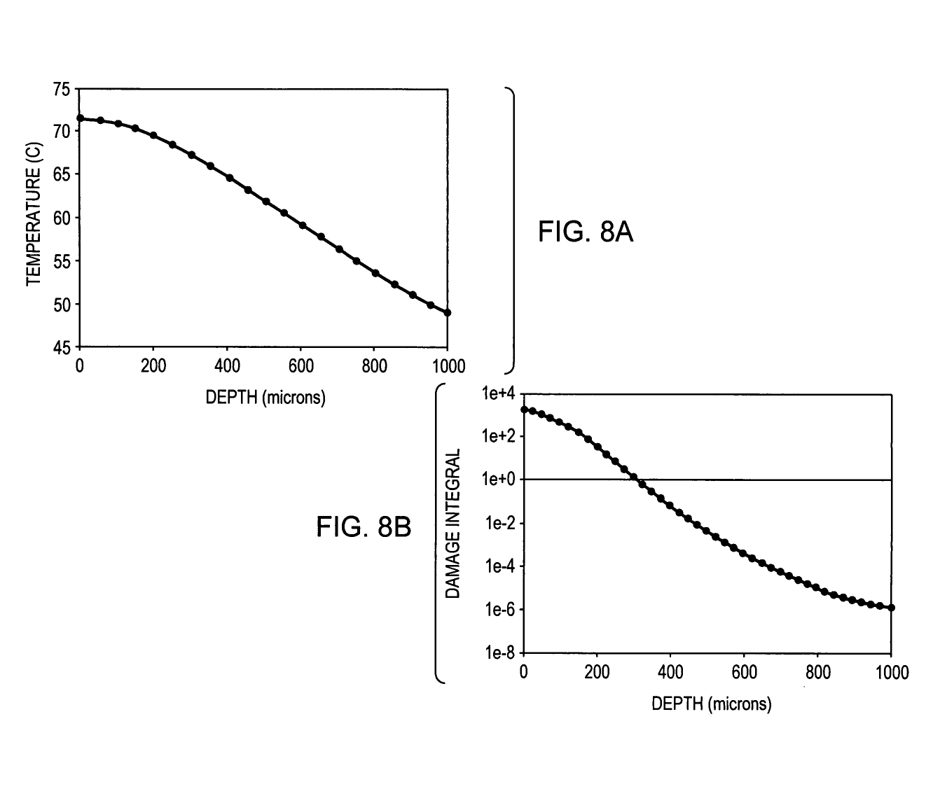

FIGS. 6A, 7A, and 8A show the temperature plotted versus depth through the center of the treated spot at the end of the laser pulse for a fluence of 12 J/cm² and irradiation times of 1 s, 2 s, and 5 s, respectively. FIGS. 6B, 7B, and 8B show the thermal damage plotted versus depth through the center of the treated spot at the end of the laser pulse for the same fluence and irradiation times.

The figures suggest that resulting damage is located a depth within about the first 300 μm from the surface. Table 2 shows the peak temperature at the surface and the depth where damage integral reaches 1, the latter being indicative of the damage depth. For the fluence of 12 J/cm², for irradiation times ranging from 0.2 to 5 s, the depth of damage is about 300 μm. As the irradiation time increases to 10 s, the depth of damage decreases to about 250 μm. This may be explained by heat diffusion during the longer exposure time. Finally, with higher fluences of 15 and 20 J/cm², depth of damage increases as expected.

In summary, the depth of penetration of the light at 1450 nm is about 439 μm. By proper selection of irradiation time and fluence, one can cause thermal damage of vascular tissue that is matched to the vein wall thickness of about 300 μm. One should keep in mind that these calculations are an approximate technique given the many simplifying assumptions made in the analysis. Still, the calculations are a valuable starting point for experiments; guide the choice of fluence, irradiation time, and irradiance; and provide an estimation of damage depth.

Ex Vivo Vascular Tissue Experiment on a Pig Aorta

A piece of pig aorta was obtained and used within four hours of sacrifice of the pig. The aorta was stored and transported at slightly over 0° C. Though the aortal wall may be different than a vein wall, both are vascular tissues and similar in many respects. The thickness of the aortal wall is 800 μm, which much thicker than that of, e.g., a saphenous vein wall. The model may be sufficient to understand the effect of various parameters on the thermal damage depth of the vein wall, however.

The inner surface of the aorta was exposed. The starting temperature of the tissue was 19° C., as opposed to desired normal body temperature of 37° C. This led to somewhat different results than that would have been obtained in vivo. An approximate correction factor was applied to take into account the different initial temperatures.

The CW laser light from laser diodes is coupled into a fiber that is then delivered through optics in a handpiece to generate a 6-mm circular collimated flat top spot at 1450 nm. Table 3 shows the parameters for multiple spots that were treated at different fluences with varying irradiation times and irradiances. A 6-mm circular punch biopsy was taken and split in the middle. The samples were fixed in 10% buffered formalin solution and processed. Vertical sections, 10 μm thick were stained with Hematoxylin and Eosin (H&E) and observed under an optical microscope. Tissue with thermal damage is stained darker. Depth of thermal damage was assessed by observation through an optical microscope fitted with a calibrated reticle. Digital photographs were also taken.

TABLE 3
Fluence, irradiation times, and damage depth for
ex vivo vascular tissue experiments
(n.d. = not determined)

| | Expt Number | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Fluence | 15 | 15 | 15 | 20 | 20 | 20 | 20 | 25 | 25 | 25 | 25 | 33 |
| (J/cm²) | | | | | | | | | | | | |
| Irradiation | 0.5 | 1.0 | 2.0 | 0.5 | 1.0 | 2.0 | 5.0 | 1.0 | 2.0 | 5.0 | 10.0 | 10.0 |
| Time (s) | | | | | | | | | | | | |
| Damage | 300 | n.d. | n.d. | 400 | 300 | 300 | error | 500 | 450 | 800 | 800 | 800 |
| Depth (μm) | | | | | | | | | | (Full) | (Full) | (Full) |

The damage depths shown in Table 3 were estimated by microscopic observation of the samples. The damage depth ranges from minimal to 300 μm to as high as 800 μm, the full thickness of the aortal wall. For a given fluence, the depth of damage is lower for longer irradiation times, probably due to the diffusion of heat during irradiation. At higher fluences or lower irradiation times or both, slight separation of the superficial vein wall fibrous material was seen. However, no audible popping sound indicating a severe explosive ablative process was noted.

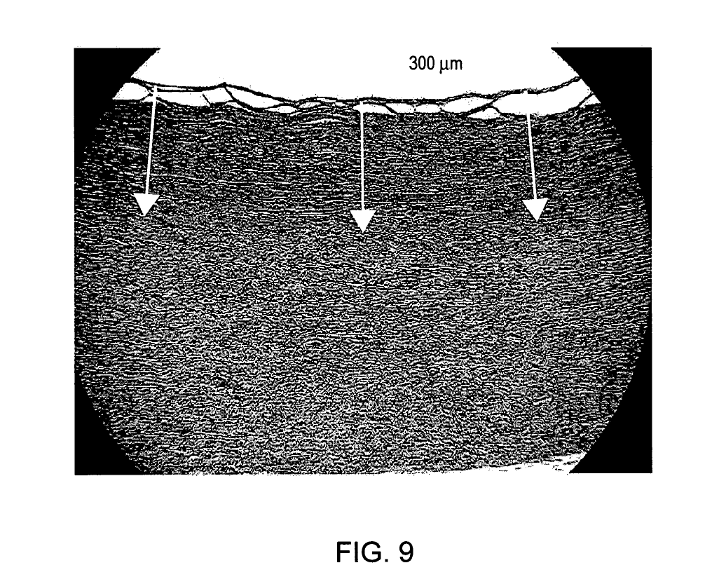

FIGS. 9-11 show photographs of histology slides of pig aortas. The arrows in each of the figures show the approximate depth of the thermal damage.

FIG. 9 shows the histology of an aorta, with an initial temperature of 19° C., after treatment with a fluence of 20 J/cm² and an irradiation time of 1 s. Damage to a depth of about 300 μm is seen.

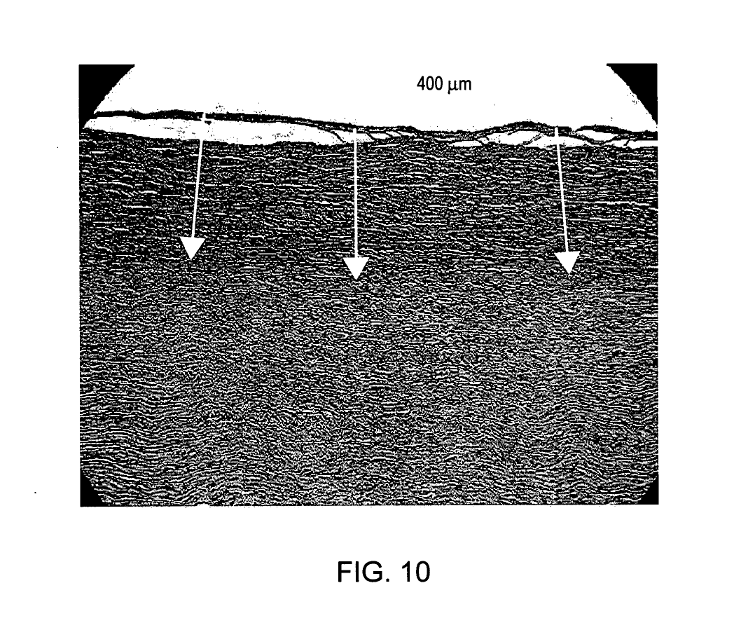

FIG. 10 shows the histology of an aorta, with an initial temperature of 19° C., after treatment with a fluence of 20 J/cm² and an irradiation time of 0.5 s. Damage to a depth of about 400 μm is seen.

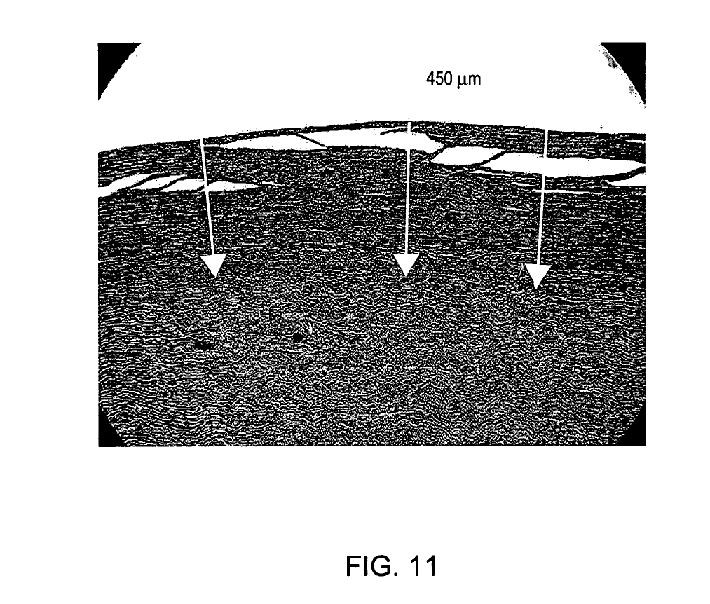

FIG. 11 shows the histology of an aorta, with an initial temperature of 19° C., after treatment with a fluence of 25 J/cm² and an irradiation time of 2 s. Damage to a depth of about 450 μm is seen.

One can see that a fluence of 15 J/cm² or higher is used for eliciting thermal damage, whereas the calculations predicted a fluence of 12 J/cm². In the calculations, the initial temperature was assumed to the normal body temperature of 37° C., whereas in the ex vivo tissue experiments of the pig aorta, the temperature was 19° C. If one were to assume that the critical temperature for thermal damage of the aortal wall is 79° C., the fluence needed to damage tissue starting at 19° C. would be a factor, [(79−19)/(79−37)], or 1.43 times higher than 12 J/cm², i.e., 17 J/cm². Fluence on the order of about 17 J/cm² caused desired thermal damage in these experiments.

In all the experiments above, no audible popping sound that would indicate an explosive ablation process was heard. This is desirable since ablation can cause vein perforations, which can lead to unwanted side effects such as bruising and resulting tenderness.

Using 1450 nm wavelength laser light and by controlling the fluence and the irradiation time, the depth of thermal damage can be controlled and matched with the vein wall thickness. A fluence of about 25 J/cm² and irradiation time of about 5 s may be appropriate for vascular tissue at 19° C. For tissue at normal body temperature, this translates to a fluence of about 17.5 J/cm².

Exemplary Experimental Treatment on a Human Leg Vein

Samples of veins from human legs were acquired from a patient who underwent an ambulatory phlebectomy. The veins were transported in saline, and the experiment was conducted within 24 hours after removal from the patient's body. A glass fiber with a 600 μm core was introduced into a vein. A light source, which consisted of a bank of laser diodes emitting at wavelengths ranging from 1440 nm to 1460 nm, was coupled into a fiber bundle. The light source was operated CW, and the light exiting from the fiber bundle was coupled into the glass fiber inserted into the vein.

The glass fiber was withdrawn at a constant rate with a pullback device powered by a stepper motor, as described in more detail above. Table 4 shows the laser powers and optical fiber withdrawal rates used for various samples. Samples of the vein after treatment, as well as untreated control specimens, were fixed in a 10% buffered formalin solution. The samples were sectioned, and cross-sections of the vein 10 μm thick were examined after H&E staining.

TABLE 4
Laser powers and optical fiber withdrawal rates
for ex vivo human vein experiments.

| Sample | Laser | Withdrawal |
|---|---|---|
| Number | Power, W | Rate, mm/s |
| 1, | NA | NA |
| Control | | |
| 2 | 1.0 | 1.5 |
| 3 | 1.5 | 1.5 |
| 4 | 2.0 | 1.5 |
| 5 | 2.5 | 1.5 |
| 6 | 1.0 | 1.0 |
| 7 | 1.5 | 1.0 |

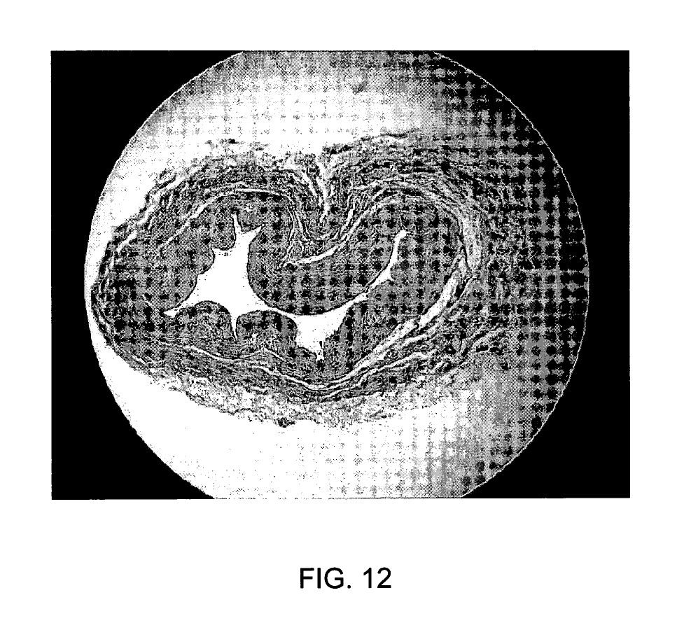

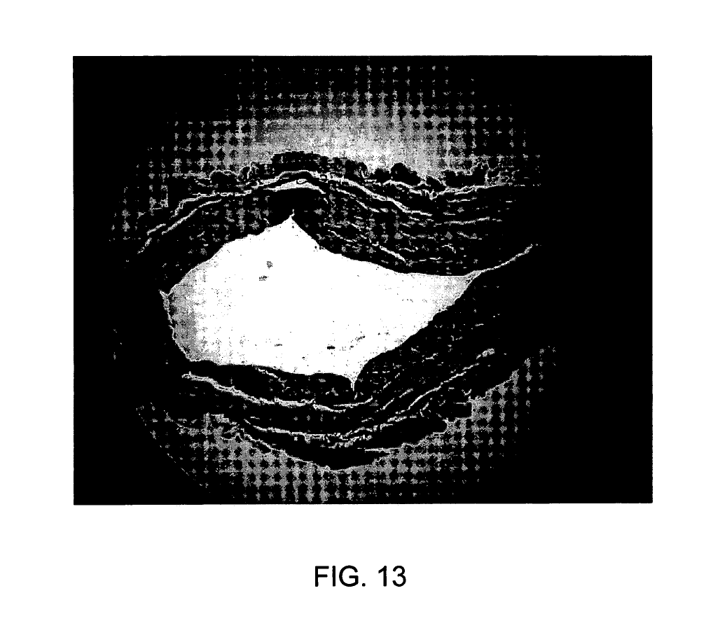

Gross visual observation indicated heating and shrinkage of the vein. Observation of the histology samples under an optical microscope indicated full thickness thermal damage and denatured connective tissue, as can be seen in FIGS. 12 and 13. FIG. 12 shows a photograph of a histology slide of a cross-section of a human vein prior to being treated with laser radiation. This is the control sample (Sample Number 1 in Table 4). FIG. 13 shows a photograph of a histology slide of a cross-section of a human vein treated according to the invention. This is Sample Number 2 according to Table 4.

While the invention has been particularly shown and described with reference to specific illustrative embodiments, it should be understood that various changes in form and detail may be made without departing from the spirit and scope of the invention as defined by the appended claims.

1. A method of treating a blood vessel, comprising:
providing a beam of light comprising a wavelength longer than about 1,160 nm; and
delivering endovascularly the beam of light to a wall of a targeted blood vessel.

2. The method of claim 1 further comprising delivering the beam of light to a target chromophore in the wall of the targeted blood vessel.

3. The method of claim 2 wherein the target chromophore comprises water.

4. The method of claim 1 further comprising delivering the beam of light via an optical fiber.

5. The method of claim 4 further comprising delivering the beam of light through a diffusing tip connectable to the optical fiber.

6. The method of claim 1 further comprising reducing the size of the targeted blood vessel.

7 The method of claim 3 further comprising heating the target chromophore to a temperature below about 80° C.

8. The method of claim 4 wherein the optical fiber is in communication with a pullback device for positioning the optical fiber.

9. The method of claim 8 wherein the pullback device withdraws the optical fiber from the targeted blood vessel at a rate of between about 0.5 mm/s and about 2 mm/s.

10. The method of claim 1 further comprising substantially removing blood from at least a portion of the targeted blood vessel before delivering endovascularly the beam of light to the wall of the targeted blood vessel.

11. The method of claim 1 wherein the beam of light has a wavelength between about 1160 nm and about 2600 nm.

12. The method of claim 11 wherein the beam of light has a wavelength between about 1300 nm and about 1560 nm.

13. The method of claim 12 wherein the beam of light has a wavelength of about 1450 nm.

14. The method of claim 11 wherein the beam of light has a wavelength of about 2100 nm.

15. The method of claim 1 wherein the beam of light has a fluence between about 3 J/cm² and about 100 J/cm².

16. The method of claim 1 wherein the beam of light has a power between about 0.5 W and about 5 W.

17. The method of claim 1 wherein the irradiation time of the beam of light is between about 0.2 s and about 10 s.

18. The method of claim 1 wherein the penetration depth of the beam of light is between 0.05 mm and about 2.0 mm.

19. The method of claim 18 wherein the penetration depth of the beam of light is about 300 μm.

20. A method of treating a blood vessel, comprising:
providing a beam of light comprising a wavelength longer than about 1,160 nm;
introducing a light-absorbing medium adjacent a wall of a targeted blood vessel, the light-absorbing medium absorbing at least one wavelength of the beam of light; and
delivering endovascularly the beam of light to the light-absorbing medium.

21. An apparatus for treating a blood vessel, the apparatus comprising:
a light source providing a beam of light comprising a wavelength longer than about 1,160 nm;
a delivery system for introducing a light-absorbing medium adjacent a wall of a targeted blood vessel; and
an optical fiber for delivering endovascularly the beam of light to the light-absorbing medium in the targeted blood vessel.

22. The apparatus of claim 21 wherein the optical fiber comprises a diffusing tip.

23. The apparatus of claim 21 wherein the optical fiber is in communication with a pullback device for positioning the optical fiber.

24. The apparatus of claim 23 wherein the pullback device withdraws the optical fiber from the targeted blood vessel at a rate of between about 0.5 mm/s and about 2 mm/s.

25. The method of claim 21 wherein the beam of light has a wavelength between about 1160 nm and about 2600 nm.

26. The apparatus of claim 25 wherein the beam of light has a wavelength between about 1300 nm and about 1560 nm.

27. An apparatus for treating a blood vessel, comprising:
means for providing a beam of light comprising a wavelength longer than about 1,160 nm;
means for introducing a light-absorbing medium adjacent a wall of a targeted blood vessel; and
means for delivering endovascularly the beam of light to the light-absorbing medium.

28. A kit for treating a blood vessel, the kit comprising:
a light source providing a beam of light comprising a wavelength longer than about 1,160 nm;
an optical fiber for delivering endovascularly the beam of light to a wall of a targeted blood vessel; and
instructions means comprising instructions for using the light source and optical fiber to improve the appearance of the targeted blood vessel by reducing its size.
